(19) United States
(12) Patent Application Publication
(10) Pub. No.: US 2008/0153094 A1
(43) Pub. Date: Jun. 26, 2008

(54) REDUCTION OF NONSPECIFIC BINDING IN NUCLEIC ACID ASSAYS AND NUCLEIC ACID SYNTHESIS REACTIONS

(76) Inventor: James M. Minor, Newark, DE (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/871,791

(22) Filed: Oct. 12, 2007

Related U.S. Application Data
(60) Provisional application No. 60/829,333, filed on Oct. 13, 2006.
(60) Provisional application No. 60/864,444, filed on Nov. 6, 2006.

(51) Int. Cl. C12Q 1/68; C07H 21/04

(52) U.S. Cl. 435/6; 536/25.3

(57) ABSTRACT

The methods of the present invention described herein may be carried out to reduce unwanted binding of probes or other nucleic acids, and thereby reduce noise (i.e., improve signal-to-noise characteristics of signals read from these probes for their intended targets) or interference in nucleic acid assays and nucleic acid synthesis reactions. The time such assays or reactions take to complete is also reduced.

The present invention relates to at least two methods: (i) methods of reducing noise and increasing efficiency in nucleic acid assays; and (ii) methods of increasing efficiency and accuracy of nucleic acid synthesis. By increasing efficiency in assays, it is meant that the reaction time to achieve a given signal is reduced and the signal strength may be increased. By increasing efficiency in nucleic acid synthesis, it is meant that greater copy numbers of accurate nucleic acids are produced in shorter periods of time.

### PRIORITY CLAIM

This application claims priority to U.S. Provisional Application No. 60/829,333, filed on Oct. 13, 2006, and U.S. Provisional Application No. 60/864,444, filed on Nov. 6, 2006.

### FIELD OF THE INVENTION

The field of the invention relates to reducing nonspecific binding of probes and thereby reduce signal noise (i.e., improve signal-to-noise characteristics of signals read from these probes for their intended targets) and increasing efficiency of nucleic acid reactions. The field of the invention also relates to reducing unwanted binding in nucleic acid synthesis reactions.

### BACKGROUND OF THE INVENTION

Unwanted interactions between nucleic acids can negatively impact both nucleic acid assays and nucleic acid synthesis. Identifiable sequenced probes are used in many useful bioassays to analyze the presence and abundance of nucleic acids in target species from a biological sample. Two well-known assays are branched DNA (bDNA) signal amplification and nucleic acid microarrays. Other nucleic acid assays that incorporate probes are northern and Southern blots.

bDNA signal amplified assays are used to measure viral load in patient blood of HIV and liver diseases for diagnostic/prognostic evaluations. Hybridization of signal amplifier components to nucleic acids in addition to the target viral nucleic acids is the primary source of signal error.

Arrays of binding agents or probes, such as polypeptide and nucleic acids, have become an increasingly important tool in the biotechnology industry and related fields. These binding agent arrays, in which a plurality of probes are positioned on a solid support surface in the form of an array or pattern, find use in a variety of different fields, e.g., genomics (in sequencing by hybridization, SNP detection (or single nucleotide polymorphism detection), differential gene expression analysis, CGH analysis (or comparative genomic hybridization analysis), location analysis, identification of novel genes, gene mapping, finger printing, etc.) and proteomics.

In using such arrays, the surface-bound probes are contacted with molecules or analytes of interest, i.e., targets, in a sample. Targets in the sample bind to the complementary probes on the substrate to form a binding complex. The pattern of binding of the targets to the probe features or spots on the substrate produces a pattern on the surface of the substrate and provides desired information about the sample. In most instances, the targets are labeled with a detectable label or reporter such as a fluorescent label, chemiluminescent label or radioactive label. The resultant binding interaction or complexes of binding pairs are then detected and read or interpreted, for example, by optical means, although other methods may also be used depending on the detectable label employed. For example, laser light may be used to excite fluorescent labels bound to a target, generating a signal only in those spots on the substrate that have a target, and thus a fluorescent label, bound to a probe molecule. This pattern may then be digitally scanned for computer analysis.

Generally, in discovering or designing probes to be used in an array, a nucleic acid is selected based on the particular gene or genetic locus of interest, where the probe nucleic acid may be as great as about 60 or more nucleotides in length, or as small as about 25 nucleotides in length or less. From the nucleic acid sequence, probes are synthesized according to various nucleic acid regions, i.e., fragments of the nucleic acids and are associated with a substrate to produce a nucleic acid array. As described above, a detectably labeled sample is contacted with the array, where targets in the sample bind to complementary probes of the array.

Gene expression, CGH and location analysis on microarrays are examples of techniques that utilize the property of nucleotides binding to their complements. One problem relating to microarray assays is nonspecific binding, where probes on the microarray bind to other nucleic acids than their targets, in addition to binding to the intended target. This increases both the noise of the signals read from the probes and time to completion of the assay. Generally, hybridized probes will exhibit nonspecific binding to nucleic acids that are not a perfect match to the probe, as well as specific binding to nucleic acids that are perfect matches to the probe. To increase the accuracy and specificity of microarray experimental results, it is important to reduce the level of nonspecific binding to probes, as nonspecific binding generates noise that obscures the signal from the targets that are specifically bonded to the probe. There is currently no known method that adequately and significantly reduces nonspecific binding during the hybridization process.

Applicants also appreciated that unwanted binding is known to reduce the accuracy and efficiency of nucleic acid synthesis. When nucleic acids are synthesized, the products of these reactions can often interfere with the reactions themselves, such as by binding to their complementary template. Therefore, there is a need for methods of reducing unwanted binding in nucleic acid synthesis reactions, as well.

### SUMMARY OF THE INVENTION

One embodiment of the invention is a method of reducing noise and increasing efficiency in a nucleic acid assay of a biological sample comprising:

(a) providing a plurality of nucleic acid strings;

(b) combining the plurality of nucleic acid strings with an assay to determine the presence, absence, or amount a target nucleic acid; and

(c) allowing the plurality of nucleic acid strings to bind nonspecifically to nucleic acids in the assay, thereby reducing noise and increasing efficiency.

In another embodiment of the invention, the nucleic acid assay is a branched DNA assay. In another embodiment of the invention, the nucleic acid assay is an assay conducted on a microarray. In another embodiment of the invention, 90% of the nucleic acid strings in the plurality of nucleic acid strings are from 8 to 70 nucleotides long.

In another embodiment of the invention, the plurality of nucleic acid strings is prepared by using restriction enzymes to digest target nucleic acid. In another embodiment of the invention, the plurality of nucleic acid strings is prepared by using restriction enzymes to digest complement of the target nucleic acid. In another embodiment, the plurality of nucleic acid strings is prepared by using restriction enzymes to digest nucleic acids in an aliquot of the biological sample.

In another embodiment, the plurality of nucleic acid strings is prepared randomly on an oligonucleotide synthesizer. In another embodiment, the plurality of nucleic acid strings is prepared on an oligonucleotide synthesizer based on a known sequence or randomly. In another embodiment, the known sequence is fragments of the target nucleic acid or complement of the target nucleic acid.

Yet another embodiment of the invention is a method of increasing the accuracy and efficiency of a nucleic acid synthesis reaction comprising:

(a) providing a plurality of nucleic acid strings;

(b) combining the plurality of nucleic acid strings with a nucleic acid synthesis reaction comprising a template nucleic acid, synthetic and/or natural nucleic acids, and an enzyme for nucleic acid synthesis; and

(c) allowing the plurality of nucleic acid strings to bind nonspecifically to the products of the nucleic acid synthesis reaction, thereby increasing the accuracy and efficiency of a nucleic acid synthesis reaction.

In one embodiment, the nucleic acid synthesis reaction comprises transcription of DNA to RNA using a polymerase. In one embodiment, the polymerase is a T7 RNA polymerase. In one embodiment, the nucleic acid synthesis reaction comprises replication of RNA, DNA, or synthetic nucleic acids. In one embodiment, the nucleic acid synthesis reaction comprises amplification of RNA, DNA, or synthetic nucleic acids. In one embodiment, the amplification reaction is PCR.

In one embodiment, the nucleic acid synthesis reaction is a reverse transcription reaction. In one embodiment, 90% of the nucleic acid strings in the plurality of nucleic acid strings are from 8 to 30 nucleotides long. In one embodiment, the plurality of nucleic acid strings is prepared by using restriction enzymes to digest the template nucleic acid. In one embodiment, the plurality of nucleic acid strings is prepared by using restriction enzymes to digest complement of the template nucleic acid. In one embodiment, the plurality of nucleic acid strings is prepared by using restriction enzymes to digest a biological sample.

In one embodiment, the plurality of nucleic acid strings is prepared randomly on an oligonucleotide synthesizer. In one embodiment, the plurality of nucleic acid strings is prepared on an oligonucleotide synthesizer based on a known sequence. In one embodiment, the known sequence is fragments of the template nucleic acid. In one embodiment, the known sequence is fragments of complement of the template nucleic acid.

These and other features of the invention will become apparent to those persons skilled in the art upon reading the details of the methods as more fully described below.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate embodiments of the invention and together with the description, serve to explain the principles of the invention.

### BRIEF DESCRIPTION OF THE DRAWINGS

The following drawings further describe by illustration the advantages and objects of the present invention. Each drawing is referenced by corresponding figure reference characters within the “DETAILED DESCRIPTION OF THE INVENTION” section to follow.

FIG. 1 is a diagram illustrating both specific and nonspecific binding in a nucleic acid assay.

FIG. 2 is a diagram showing the invention concept for reducing nonspecific binding in a nucleic acid assay according to the present invention.

FIG. 3 is a diagram showing a method to create nucleic acid strings according to the present invention.

FIG. 4 is a diagram of a nucleic acid synthesis reaction.

FIG. 5 is a diagram showing unwanted nucleic acid binding (specifically indirect interference) in a nucleic acid synthesis reaction.

FIG. 6 is a diagram showing unwanted nucleic acid binding (specifically direct interference) in a nucleic acid synthesis reaction.

FIG. 7 is a diagram showing unwanted nucleic acid binding in a nucleic acid synthesis reaction.

FIG. 8 is a diagram showing the invention concept for reducing unwanted nucleic acid binding in a nucleic acid synthesis reaction.

### DETAILED DESCRIPTION OF THE INVENTION

### Definitions

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and PNA (peptide nucleic acids) and other polynucleotides as described in U.S. Pat. No. 5,948,902.

An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups).

A nucleotide “probe” means a nucleotide which hybridizes in a specific manner to a nucleotide target (e.g., a consensus region or an expressed transcript of a gene of interest).

“Noise” is defined as the component of an assay signal generated by nonspecific interactions of nucleic acids in the assay; it is also referred to as measurement interference or background.

A “biological sample” is a sample derived from any organism (living or dead), including animal, plant, microorganism, or virus, and may more specifically be derived from a tissue sample, such as a biopsy sample, normal tissue, blood, urine, semen, cell preparations, and the like. Biological sample also includes samples from multiple organisms that have been combined. In one embodiment, a biological sample is derived from a mammal; in another embodiment, from a human.

A “microarray” includes any one-, two- or three-dimensional arrangement of addressable regions bearing a particular chemical moiety or moieties associated with that region. A microarray is “addressable” in that it has multiple regions of moieties such that a region at a particular predetermined location on the microarray will detect a particular target or class of targets (although a feature may incidentally detect non-targets of that feature). Microarray features are typically, but need not be, separated by intervening spaces. In the case of a microarray, the “target” will be referenced as a moiety in a mobile phase, to be detected by probes, which are bound to the substrate at the various regions. However, either of the “target” or “target probes” may be the one, which is to be evaluated by the other.

Methods to fabricate microarrays are described in detail in U.S. Pat. Nos. 6,242,266; 6,232,072; 6,180,351; 6,171,797 and 6,323,043. Other drop deposition methods can be used for fabrication, as previously described herein. Also, instead of drop deposition methods, photolithographic microarray fabrication methods may be used. Interfeature areas need not be present particularly when the microarrays are made by photolithographic methods as described in those patents.

Following receipt by a user, a microarray will typically be exposed to a sample and then read. Reading of a microarray may be accomplished by illuminating the microarray and reading the location and intensity of resulting fluorescence at multiple regions on each feature of the microarray. For example, a scanner that may be used for this purpose is the AGILENT MICROARRAY SCANNER manufactured by Agilent Technologies (Palo Alto, Calif.) or other similar scanner. Other suitable apparatus and methods are described in U.S. Pat. Nos. 6,518,556; 6,486,457; 6,406,849; 6,371,370; 6,355,921; 6,320,196; 6,251,685 and 6,222,664. Scanning typically produces a scanned image of the microarray which may be directly inputted to a feature extraction system for direct processing and/or saved in a computer storage device for subsequent processing. However, microarrays may be read by any other methods or apparatus than the foregoing, other reading methods including other optical techniques or electrical techniques (where each feature is provided with an electrode to detect bonding at that feature in a manner disclosed in U.S. Pat. Nos. 6,251,685, 6,221,583 and elsewhere). In any case, detection is made for the purpose of identifying and quantifying of the particular target(s) bonded (i.e., hybridized) to a particular probe.

A microarray is “addressable” when it has multiple regions of different moieties, i.e., features (e.g., each made up of different oligonucleotides) such that a region (i.e., a “feature” or “spot” of the microarray) at a particular predetermined location (i.e., an “address”) on the microarray will detect a particular solution phase nucleic acid. Microarray features are typically, but need not be, separated by intervening spaces.

In the case of a microarray in the context of the present application, the “target” may be referenced as a moiety in a mobile phase (typically fluid), to be detected by “probes” which are bound to the substrate at the various regions.

A “scan region” refers to a contiguous (preferably, rectangular) area in which the microarray spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exists intervening areas that lack features of interest.

A “microarray layout” refers to one or more characteristics of the features, such as feature positioning on the substrate, one or more feature dimensions, and an indication of a moiety at a given location. “Hybridizing” and “binding,” with respect to nucleic acids, are used interchangeably.

A “design file” is typically provided by a microarray manufacturer and is a file that embodies all the information that the microarray designer from the microarray manufacturer considered to be pertinent to microarray interpretation. For example, Agilent Technologies supplies its microarray users with a design file written in the XML language that describes the geometry as well as the biological content of a particular microarray.

A “grid template” or “design pattern” is a description of relative placement of features, with annotation. A grid template or design pattern can be generated from parsing a design file and can be saved/stored on a computer storage device. A grid template has basic grid information from the design file that it was generated from, which information may include, for example, the number of rows in the microarray from which the grid template was generated, the number of columns in the microarray from which the grid template was generated, column spacings, subgrid row and column numbers, if applicable, spacings between subgrids, number of microarrays/hybridizations on a slide, etc. An alternative way of creating a grid template is by using an interactive grid mode provided by the system, which also provides the ability to add further information, for example, such as subgrid relative spacings, rotation and skew information, etc.

“Image processing” refers to processing of an electronic image file representing a slide containing at least one microarray, which is typically, but not necessarily in TIFF format, wherein processing is carried out to find a grid that fits the features of the microarray, e.g., to find individual spot/feature centroids, spot/feature radii, etc. Image processing may even include processing signals from the located features to determine mean or median signals from each feature and may further include associated statistical processing. At the end of an image processing step, a user has all the information that can be gathered from the image.

“Post processing” or “post processing/data analysis,” sometimes just referred to as “data analysis” refers to processing signals from the located features, obtained from the image processing, to extract more information about each feature. Post processing may include but is not limited to various background level subtraction algorithms, dye normalization processing, finding ratios, and other processes known in the art.

“Feature extraction” may refer to image processing and/or post processing, or just to image processing. An extraction refers to the information gained from image processing and/or post processing a single microarray.

“Stringency” is a term used in hybridization experiments to denote the stress on the bond between the probe and the target hybridized thereto. The bond strength/stability increases with degree of homology between the probe and the target hybridized thereto. The higher the stringency, the higher the percent homology between the probe and target necessary for a stable bond. Hybridization stringency increases with temperature and/or chemical properties such as the amounts of salts and/or formamide in the hybridization solution during a hybridization process. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. One example of stringent hybridization conditions is hybridization in 6× sodium chloride/sodium citrate (SSC) at about 45° C., followed by at least one wash in 0.2×SSC, 0.1% SDS at 50° C. A second example of stringent hybridization conditions is hybridization in 6×SSC at about 45° C., followed by at least one wash in 0.2×SSC, 0.1% SDS at 55° C. Another example of stringent hybridization conditions is hybridization in 6×SSC at about 45° C., followed by at least one wash in 0.2×SSC, 0.1% SDS at 60° C. A further example of stringent hybridization conditions is hybridization in 6×SSC at about 45° C., followed by at least one wash in 0.2×SSC, 0.1% SDS at 65° C. High stringent conditions include hybridization in 0.5M sodium phosphate, 7% SDS at 65° C., followed by at least one wash at 0.2×SSC, 1% SDS at 65° C.

The “northern blot” is a technique used to study gene expression by detecting RNA in a biological sample, using electrophoresis and a hybridization probe. The “Southern blot” is a technique for detecting DNA in a biological sample, using electrophoresis and a hybridization probe. Both of these techniques are well described in the art.

Methods recited herein may be carried out in any order of the recited events which is logically possible, as well as the recited order of events.

### I. Methods of Reducing Unwanted Binding in Nucleic Acid Assays and Nucleic Acid Synthesis Reactions

The methods of the present invention described herein may be carried out to reduce unwanted binding of probes or other nucleic acids, and thereby reduce noise (i.e., improve signal-to-noise characteristics of signals read from these probes for their intended targets) or interference in nucleic acid assays and nucleic acid synthesis reactions. The time such assays or reactions take to complete is also reduced.

The present invention relates to at least two methods: (i) methods of reducing noise and increasing efficiency in nucleic acid assays; and (ii) methods of increasing efficiency and accuracy of nucleic acid synthesis. By increasing efficiency in assays, it is meant that the reaction time to achieve a given signal is reduced and the signal strength may be increased. By increasing efficiency in nucleic acid synthesis, it is meant that greater copy numbers of nucleic acids are produced in shorter periods of time.

Using the techniques of the invention, unwanted binding in nucleic acid assays and nucleic acid synthesis reactions can be reduced by 10%, 20%, 30%, 40%, 50%, 60%, 70%, 80%, 90%, 95%, 99%, and 100%, with corresponding percentage increases in efficiency, reduction of noise in assays, and increased accuracy in nucleic acid synthesis.

A. Methods of Reducing Noise and Increasing Efficiency in Nucleic Acid Assays

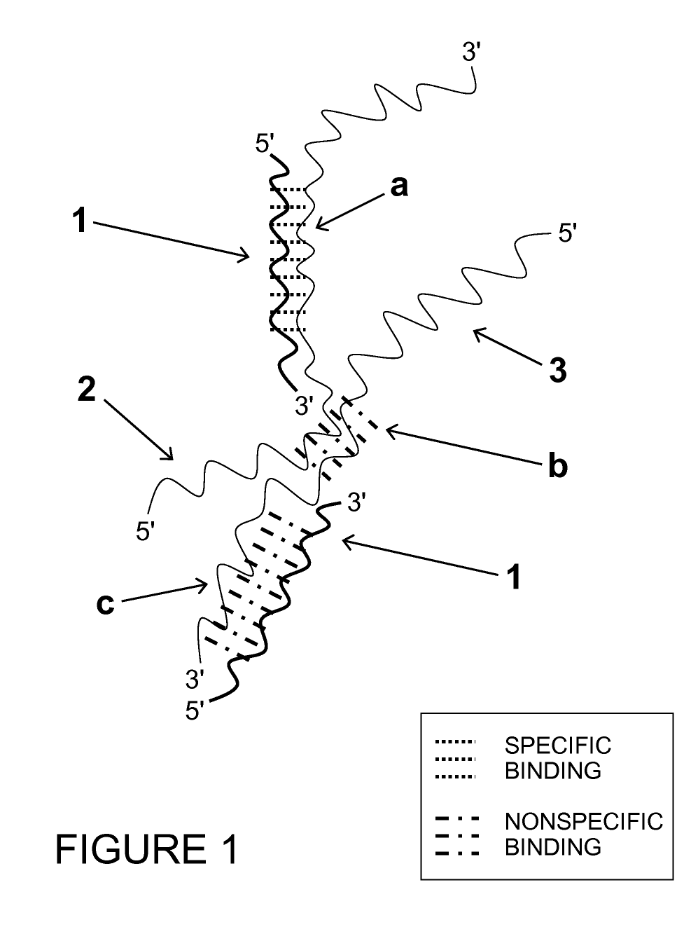

Nonspecific binding of nucleic acids can cause signal noise and reduced efficiency in nucleic acid assays. FIG. 1 illustrates the deleterious effect of nonspecific binding in a nucleic acid assay. FIG. 1 illustrates the binding of a probe 1 for its target 2; however, it also shows the nonspecific binding of target 2 to nontarget 3, as well as the nonspecific binding of the probe 1 to nontarget 3. It is one of the objects of this invention to reduce or eliminate the unwanted nonspecific binding shown in FIG. 1.

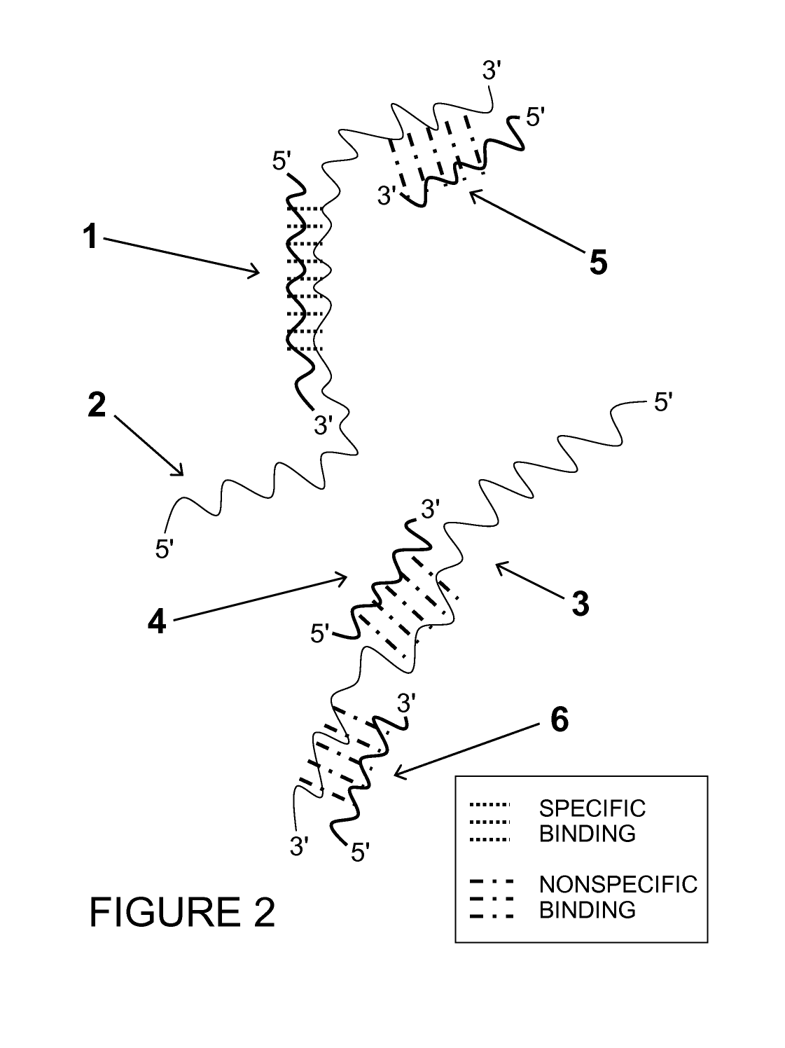

One method of achieving this objective uses a plurality of nucleic acid strings, such as reference numbers 4, 5, and 6 in FIG. 2, that inhibit deleterious nonspecific binding of probes to non-target nucleic acids (bond c in FIG. 1) and of target nucleic acids to non-target nucleic acids (bond b in FIG. 1). In one embodiment, the plurality of nucleic acid strings, such as 4, 5, and 6 in FIG. 2, function by binding to two different nucleic acids: target 2, specific to probe 1, and miscellaneous nontarget 3. This binding blocks both nonspecific binding of probe 1 to nontarget 3 (and reduces assay signal (i.e., noise) that would have been generated by this nonspecific binding), as well as nonspecific binding of target 2 to nontarget 3, which could reduce signal strength (by potentially preventing binding of probe 1 to target 2). This allows the maximum signal strength to be produced by the binding of probe 1 to target 2, and reduces the noise created by nonspecific binding of probe 1 to non-target 3. It also ensures nontarget 3 can pursue its specific probe (not shown), if it exists, so that diffusion becomes faster. Consequently, the intended hybridization reactions are enabled to produce high-fidelity measurements of each probe-specific target in the sample.

For example, nontarget 3 can be the target of another probe or it can be nucleic acids that are not being detected during the assay. In one embodiment, nontarget 3 can be nucleic acids from a biological sample, such as blood or tissue of a patient. In another embodiment, nontarget 3 can be the target of another probe on a microarray.

For example, a branched DNA or bDNA assay could be conducted according to this method. Branched DNA is a sandwich nucleic acid hybridization procedure that may be performed on a biological sample to detect target nucleic acids (such as, but not limited to, viral nucleic acids), and is described in Collins, et al., A branched DNA signal amplification assay for quantification of nucleic acid targets below 100 molecules/ml, Nucleic Acids Research, 25(15): 2979-2984 (1997). Viral RNA released from virions is captured to a microwell by a set of specific, synthetic oligonucleotide capture probes (and/or capture extender probes). A set of label extender probes hybridizes to both the viral RNA and preamplifier probes. The capture probes and the label extender probes bind to different regions of the viral RNA genome. Preamplifier probes bind to the label extender probes. Amplifier probes hybridize to the pre-amplifier probes, forming a branched DNA complex. Multiple copies of an alkaline phosphatase (AP) labeled probe specific for the amplifier probes is then hybridized to the complex. Detection is achieved by incubating the AP-bound complex with a chemiluminescent substrate, and light emission, directly related to the amount of viral RNA present, is measured. Multiple embodiments of bDNA technology are known in the art and this invention is believed to function to improve all of them. In the example of a bDNA assay, there are a plurality of probes used to capture and detect the target nucleic acids (such as the viral nucleic acids), and thus it is even more important that nonspecific binding of probes is reduced. bDNA assays may be used to detect a variety of nucleic acids including nucleic acids from viruses such as HIV, hepatitis C or hepatitis B.

In another embodiment, the present invention reduces noise and increases the efficiency of nucleic acid assays which are conducted on a microarray. In microarray technology, a plurality of known probes is affixed to specific locations on a microarray device. A biological sample is then prepared by partially digesting nucleic acids and preparing a labeled target. In one embodiment, mRNA is isolated, a reverse transcriptase used with labeled nucleotides, and labeled cDNA (i.e., target) is prepared. In another embodiment, mRNA is isolated and reverse transcriptase is used to prepare unlabeled cDNA, then labeled cRNA is synthesized from the double stranded cDNA using T7 RNA polymerase in the presence of labeled nucleotides creating labeled cRNA (i.e., target). Multiple biological samples can be processed on the same microarray. The labeled target is then hybridized to the microarray and the labeled nucleic acids are detected. As shown in the Figures, and discussed above, the nucleic acid strings of the invention are expected to block nonspecific binding of target nucleic acids to nontarget nucleic acids, and probes to nontarget nucleic acids. Microarray technology is described in Agilent Low RNA Input Linear Amplification Kit Protocol, Version 4.0, Manual Part No. 5185-5818 (January 2006) and microarrays are available from Agilent Technologies, Inc. (Wilmington, Del.).

Instead of traditional microarrays, alternative technologies such as the BeadArray™ technology available from Illumina® Inc. (San Diego, Calif.) may be used in combination with the present invention. Additionally, alternative technologies such as VeraCode™ microbeads available from Illumina® Inc. (San Diego, Calif.) may be used in combination with the present invention.

In one embodiment, the nucleic acid strings are added to the assay immediately before or during the labeling step of any assay. Therefore, for example, in a microarray experiment, the nucleic acid string can be added during or immediately before the preparation of the labeled target. Alternatively, in another embodiment, the nucleic acid strings are added to the assay immediately before or when the probe is brought into contact with the target. In a microarray experiment, this is when the target is applied to the microarray. In a bDNA assay, the nucleic acid strings may be applied at any of the steps, with the optimal effect achieved by adding the nucleic acid strings when the target nucleic acid is first exposed to the capture probes.

The nucleic acid strings may be applied at such concentrations so as to reduce nonspecific binding by the percentages described above. For example, concentrations of 100 to 2000, 200 to 800, 400 to 600, or 500 ng/μl.

These methods are expected to function equally well in any nucleic acid assay using a probe to detect a target. For instance, other nucleic acid assays that incorporate probes are northern and Southern blots, for the detection of RNA and DNA, respectively, and it is expected that the present method would improve these assays, as well.

B. Methods of Increasing Efficiency and Accuracy of Nucleic Acid Synthesis

The invention also encompasses methods of increasing efficiency and accuracy of nucleic acid synthesis. The reaction times for such synthesis reactions are likewise shortened. Specifically, the methods may be used to improve the efficiency and accuracy of synthesis of nucleic acids including, but not limited to, DNA, RNA, or synthetic nucleic acids.

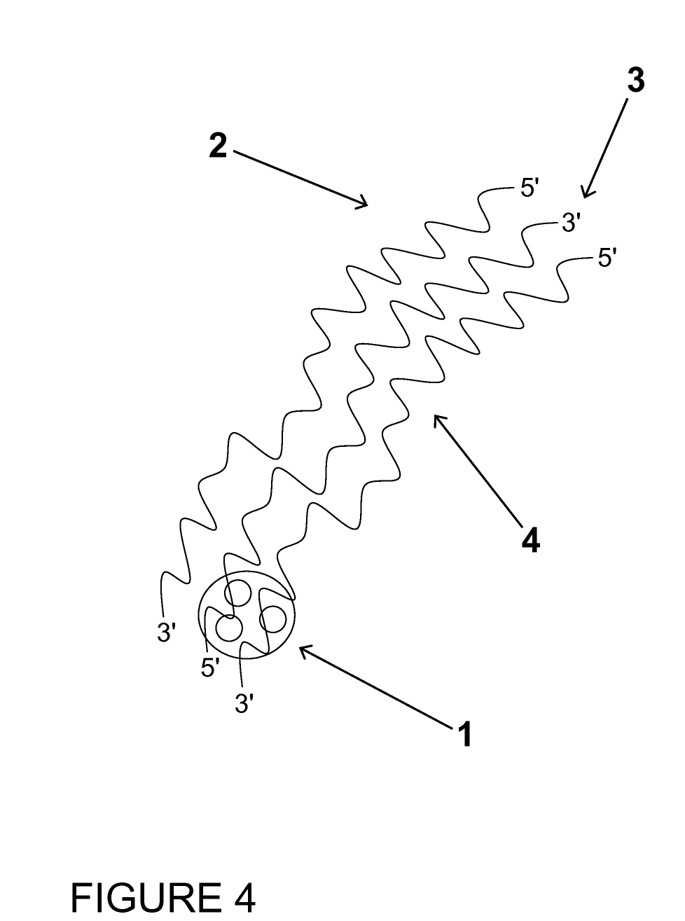

Specifically, FIGS. 4-8 illustrate reactions used to transcribe double stranded DNA into RNA, using an RNA polymerase, such as the T7 RNA polymerase, and methods of increasing efficiency and accuracy of this reaction. A number of interactions between the synthesized RNA and the double stranded DNA can occur, interfering with transcription of additional copies of the RNA. The present invention directly addresses and reduces those interactions, allowing for more accurate RNA transcription at greater copy number. The figures are based on such typical DNA and RNA strings with the 3′ and 5′ ends as indicated.

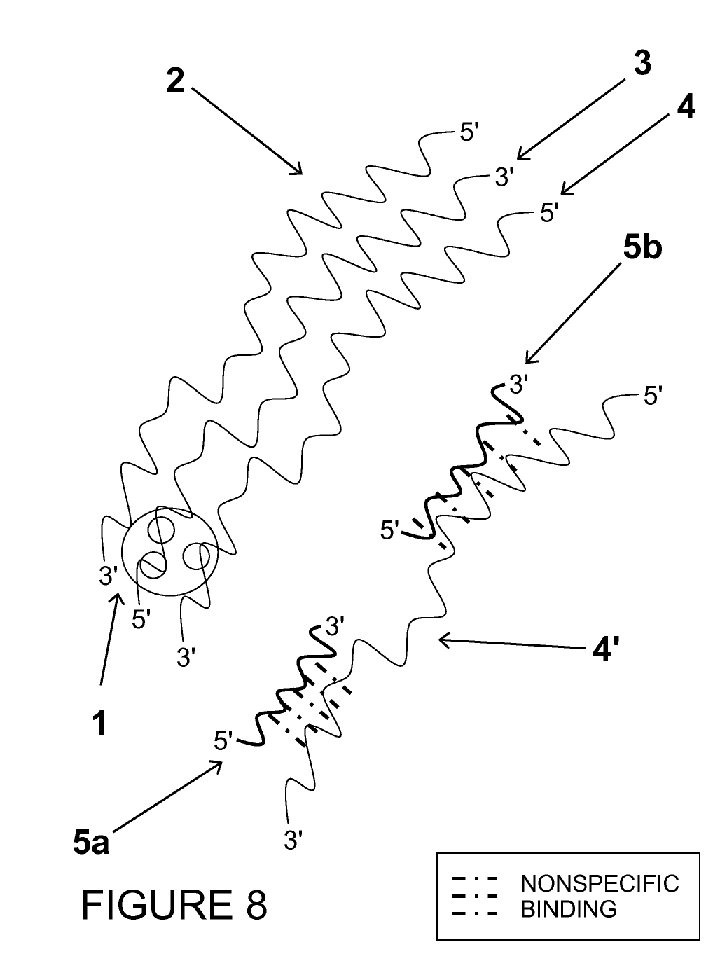

One embodiment of the present invention uses a plurality of nucleic acid strings 5a and 5b (FIG. 8) to inhibit deleterious processes induced by the increasing number of completed complementary copies 4′ that interfere with the synthesis of additional copies, causing both copy errors and premature copy termination (FIGS. 4-7). The plurality of nucleic acid strings (shown illustratively as 5a and 5b) is complementary to the copies 4′; thereby, reducing the affinity of copies 4′ for the template 3 and hence preventing the deleterious interference processes. Consequently, the synthesis reactions are enabled to produce a population of accurate complementary copies of the original template at high copy numbers. The plurality of nucleic acid strings (5a and 5b) must itself not interfere significantly with the synthesis process or with any subsequent applications of the final population of complementary copies, such as biological assay hybridizations and their signal measurements. Alternatively, the plurality of nucleic acid strings and/or any inadvertent amplifications of the same, may be removed using standard techniques based on the difference in size between the larger complementary copies 4′ and the shorter plurality of nucleic acid strings 5a and 5b, such as via gel electrophoresis or size exclusion chromatography.

FIG. 4 illustrates a hypothetical RNA synthesis process using double stranded DNA as a template. In this Figure, 3 is a DNA template and 1 is the polymerase complex, including T7 RNA polymerase, which copies each residue to its RNA complement, as it produces the complement RNA 4. Note that generally the template 3 is attached to its DNA complement 2 in a double-stranded helix configuration. The T7 polymerase initiates copying by a primer/promoter complex. The DNA helix is locally separated (unzipped) by the polymerase as it copies each template residue. The DNA then closes (re-zips) as the polymerase passes.

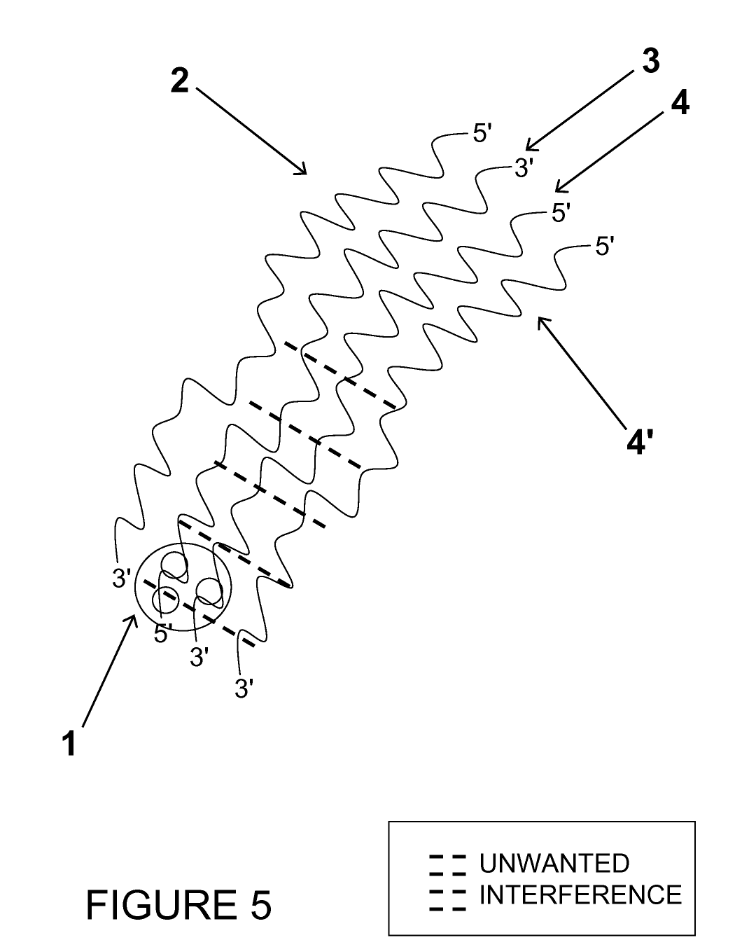

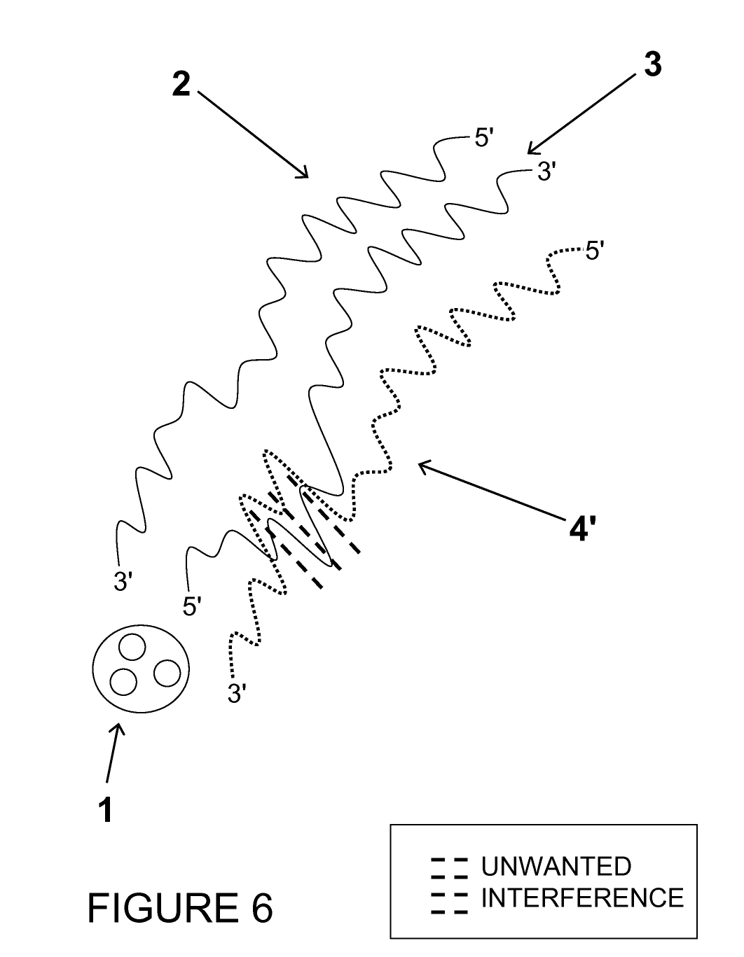

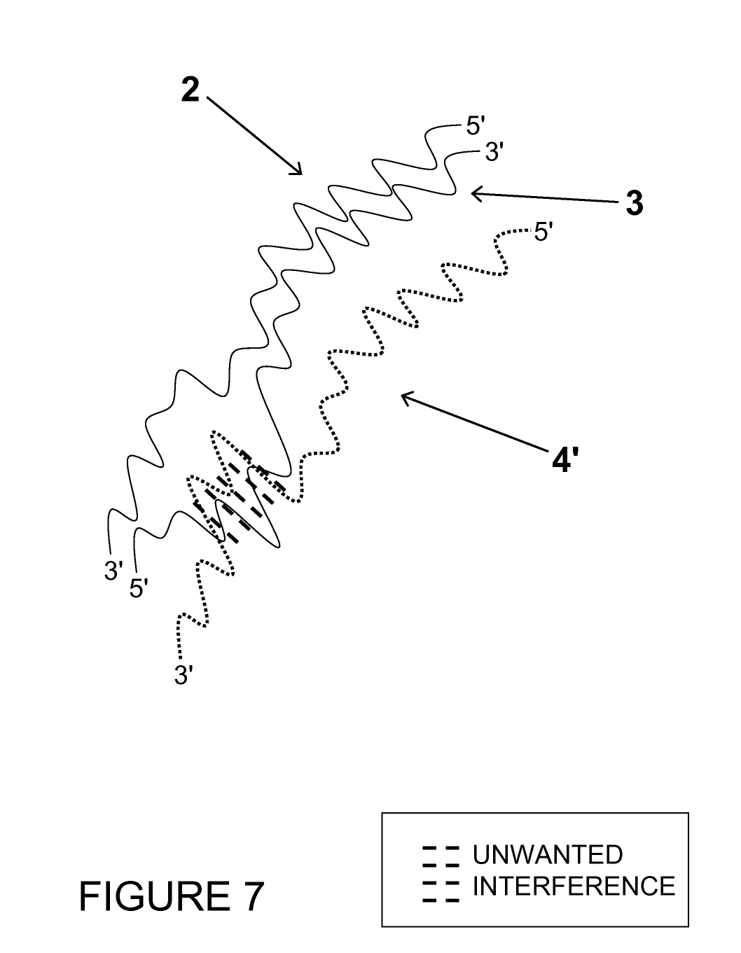

FIG. 5 illustrates unwanted interference by a completed RNA copy 4′ that is attracted to its matched template 3. The interference impacts the complementary chemical (nucleotide) affinities of template 3, which affects the coupling efficiency of the polymerase increasing the probability of copy errors and premature copy termination. Even worse, the unzipped DNA exposes the template to the RNA 4′ so that a two-strand RNA-DNA or three-strand DNA-DNA-RNA complex is formed which will interfere with the transcription process. Such a three-strand “braided” complex could form as shown in FIGS. 6 and 7. These complexes become more likely as the concentration of RNA copies 4′ increases.

FIG. 8 illustrates how the invention prevents formation of these deleterious complexes by intercepting the RNA copies 4′ with nucleic acid string 5a and 5b.

The invention also encompasses methods of increasing efficiency and accuracy of other forms of nucleic acid synthesis. For instance, these techniques can be used to improve the efficiency and accuracy of replication, amplification, reverse transcription, or any other type of nucleic acid synthesis. In one specific embodiment, the method can be used to improve the efficiency of PCR (polymerase chain reaction). In all of these synthesis reactions, the increasing copy number of the product can interfere with the synthesis of more product and the invention is used to reduce that interference. As the details of these nucleic acid synthesis reactions are well known in the art, the skilled artisan will be able to apply the present methods to these other synthesis reactions, as well.

In one embodiment, the nucleic acid strings are added prior to the initiation of the nucleic acid synthesis reaction. In another embodiment, the nucleic acid strings are added at the initiation of the nucleic acid synthesis reaction.

The nucleic acid strings may be applied at such concentrations so as to reduce nonspecific binding by the percentages described above. For example, concentrations of 100 to 2000, 200 to 800, 400 to 600, or 500 ng/μl.

### II. Methods of Making Nucleic Acid Strings

Nucleic acid strings for use in the methods of this invention can be prepared easily using known recombinant biology techniques. For both (i) methods of reducing noise and increasing efficiency in nucleic acid assays and (ii) methods of increasing efficiency and accuracy of nucleic acid synthesis, nucleic acid strings can be made either by digesting nucleic acids into short strings and, if those strings are double stranded, unannealing them to form single stranded strings or constructing short nucleic acid strings on an oligonucleotide synthesizer. Nucleic acid strings can either be produced randomly or they can be produced from nucleic acids present in the assays, such as the target nucleic acid in an assay reaction or the template nucleic acid in a synthesis reaction, or the complements of the target or template. They may also be produced from nontarget or nontemplate nucleic acids in a sample, such as patient nucleic acids.

A. Nucleic Acid Strings for Nucleic Acid Assays

The nucleic acid strings must not interfere significantly with other assay process such as biological assay signal production and signal measurements/extractions. Specifically, the plurality of nucleic acid strings comprise unlabeled diverse nucleic acids, such as 4, 5, and 6 in FIG. 2, which can nonspecifically bind to the target or nontarget nucleic acids in a sample. The nucleic acid strings can be designed to avoid significant interference with specific probe-target hybridization by making their length short and/or by designing their compositions to avoid the specific probes. Likewise, in one embodiment, the nucleic acid strings are unlabeled to avoid any consequential effects in assay measurements, i.e., signal production/extraction.

For example, FIG. 2 illustrates how the invention prevents formation of these deleterious complexes with a population of complementary nucleic acid strings, such as 4, 5, and 6. The nucleic acid strings are sufficiently short and/or do not have the capacity for competitive specific binding to interfere with the specific intended target-probe hybridizations. Hence, the ideal hybridization process is protected and maintained. Note the population (i.e., plurality) of nucleic acid strings must be adequate in both diversity and numbers to adequately protect all nucleic acids in the population of targets.

Specifically, for nucleic acid assays, nucleic acid strings should be sufficiently long to bind nonspecifically to the nucleic acids in the assay, and should therefore have a minimum length of 5, 8, 10, 12, 15, 18, or 20 base pairs. The maximum length of the nucleic acid strings may be 15, 18, 20, 25, 28, 30, 35, 40, 45, 50, 55, 60, 65, or 70 base pairs. Ranges may be formed by any combination of a lower minimum length number and a higher maximum length number. Additionally, in one embodiment, the nucleic acid strings are shorter than the probes being used in the assay. The length of the nucleic acid strings can also vary depending on the stringency of the assay conditions (both based on temperature and assay components, buffers, etc.), with longer strings being more useful in higher stringency assays and shorter strings being more useful in lower stringency assays.

In one embodiment, 100% of the individual nucleic acid strings in the plurality of nucleic acid strings is within the recommended length range, in another embodiment, 99%, 98%, 97%, 96%, 95%, 94%, 92%, 91%, 90%, 85%, 80%, 75%.

In one embodiment, the strings are comprised so that nucleic acid fragments of the target are present in the strings. In another embodiment, the strings are comprised so that nucleic acid fragments of the complement of the target are present in the strings. In another embodiment, the strings are comprised so that nucleic acid fragments nontarget nucleic acids are present in the strings, such as targets for other probes or nucleic acids from an aliquot of the biological sample are present in the strings. In yet other embodiments, the nucleic acids are generated randomly. Mixtures of these types of strings may also be used. For example, a biological sample applied to a microarray could contain nucleic acid targets of multiple probes as well as nontarget nucleic acids, and a mixture of all of these could be overdigested to prepare the strings of the invention. As the present invention can be used for many nucleic acid assays, the nucleic acid strings may be composed of RNA or DNA, or synthetic nucleic acids, as the assay requires.

B. Nucleic Acid Strings for Nucleic Acid Synthesis

Likewise, for nucleic acid synthesis, the nucleic acid strings 5a and 5b of FIG. 8 must not interfere significantly with the nucleic acid synthesis or with any subsequent applications of the final population of copies 4′, such as biological assay hybridizations and their signal measurements.

Specifically, for nucleic acid synthesis, nucleic acid strings should be sufficiently long to bind nonspecifically to the nucleic acids in the assay, and should therefore have a minimum length of 5, 8, 10, 12, 15, 18, or 20 base pairs. The maximum length of the nucleic acid strings may be 15, 16, 18, 20, 25, 28, 30 base pairs. Ranges may be formed by any combination of a lower minimum length number and a higher maximum length number. Additionally, in one embodiment, the nucleic acid strings are sufficiently short so that nucleic acids are not synthesized using the strings as a template. The length of the nucleic acid strings can also vary depending on the stringency of the synthesis conditions (both based on temperature and assay components, buffers, etc.), with longer strings being more useful in higher stringency synthesis conditions and shorter strings being more useful in lower stringency synthesis conditions.

In one embodiment, 100% of the individual nucleic acid strings in the plurality of nucleic acid strings is within the recommended length range, in another embodiment, 99%, 98%, 97%, 96%, 95%, 94%, 92%, 91%, 90%, 85%, 80%, 75%.

Specifically, in FIG. 8, for template 3, the nucleic acid string 5a and 5b can be a plurality of nucleic acid strings that have adequate chemical affinity to bind nonspecifically to the completed template copies 4′ but not for template 3. For example the nucleic acid strings 5a and 5b can contain nucleic acids complementary to nucleic acids within the copies 4′. The nucleic acid string 5 can be designed to avoid participation in the copying process by making their length short and/or designing their compositions to avoid primer/promoter bindings. Likewise, the nucleic acid strings 5a and 5b can be designed to avoid any consequential effects in subsequent applications, i.e., binding with hybridization probes and/or producing assay measurement signals. Note the population of nucleic acid strings must be adequate in both diversity and numbers to adequately protect all unique nucleic acids in the population of templates from their complementary copies 4′.

In one embodiment, the strings are comprised so that nucleic acid fragments of the template nucleic acid are present in the strings. In another embodiment, the nucleic acid strings are comprised so that nucleic acid fragments of the complement of the template nucleic acid is present in the strings (such as for a replication process). In another embodiment, the nucleic acid strings are generated randomly. In an additional embodiment, the nucleic acid strings are comprised of fragments of nucleic acids present in a biological sample. Mixtures of these types of strings may also be used. As the present invention can be used for all forms of nucleic acid synthesis, the nucleic acid strings may be composed of RNA or DNA, or synthetic nucleic acids, as the synthesis reaction requires.

C. Nucleic Acid Strings can be Prepared by Restriction Digestion

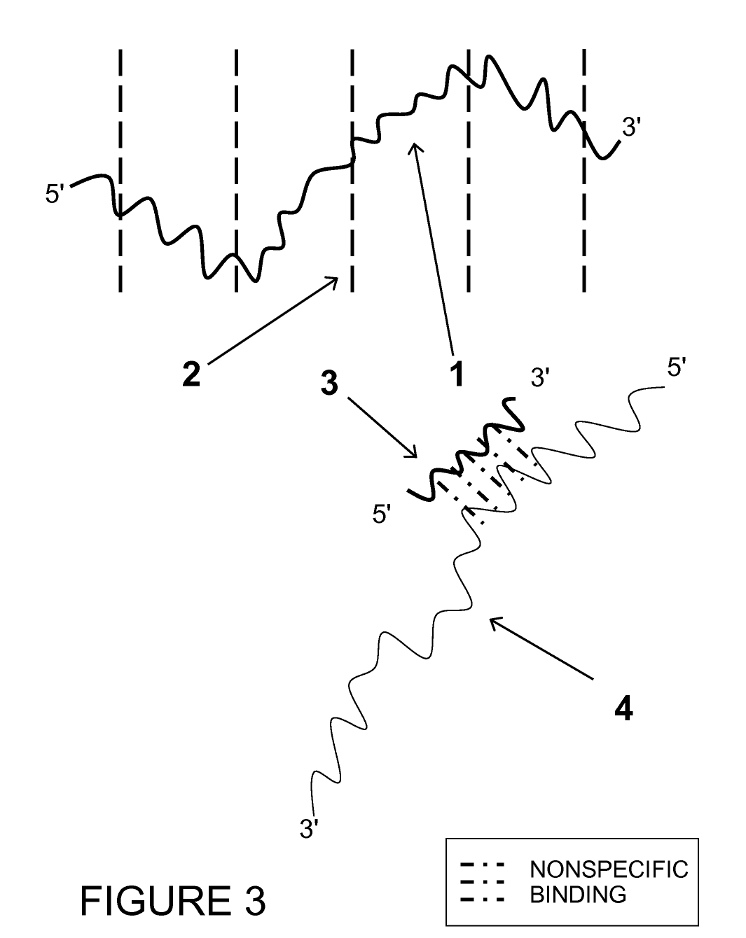

FIG. 3 shows a method to create a plurality of nucleic acid strings for the present invention. Restriction enzymes may be used to digest nucleic acids, as discussed in sections II.A and II.B, above to create nucleic acid strings of appropriate lengths. Restriction enzymes are enzymes that make specific cuts in DNA, and multiple restriction enzymes can be used at a single time to cut a longer DNA into shorter pieces. Double stranded pieces can be unannealed (unzipped) using known techniques in the art, such as heat, to form single stranded nucleic acid strings. The length of the shorter pieces (i.e., the nucleic acid strings of the present invention) can be monitored using well known techniques such as gel electrophoresis or size exclusion chromatography. In FIG. 3, 1 shows the nucleic acid string produced by the restriction digest, and 2 shows the restriction enzyme digestion site.

D. Nucleic Acid Strings can be Prepared Using Oligonucleotide Synthesis

Additionally, RNA, DNA, and synthetic nucleic acid strings can be prepared using oligonucleotide synthesis. Once the sequence or sequences of the desired population of strings is known, it is well within the skill of the person of ordinary skill in the art to prepare such strings using known oligonucleotide synthesis techniques. This technique may also be used to prepare random nucleic acid strings. Oligonucleotide synthesis refers to the non-biological, chemical synthesis of nucleic acids. Automated synthesizers allow the synthesis of a wide variety of oligonucleotides in differing lengths. Such synthesizers are available commercially from companies like BioAutomation™ (Plano, Tex.), which manufactures the MerMaid™ line of synthesizers. Oligonucleotide synthesis is also available from vendors, who will prepare oligonucleotides based on the customer's specifications. Vendors include the Midland Certified Reagent Company, Inc. (Midland, Tex.).

Therefore, once the sequence of desired strings has been identified, it is very easy to prepare them.

It is further intended that any other embodiments of the present invention that result from any changes in application or method of use or operation, method of manufacture, shape, size, or material which are not specified within the detailed written description or illustrations contained herein yet are considered apparent or obvious to one skilled in the art are within the scope of the present invention.

### EXAMPLES

### Example 1

### Adding Nucleic Acid Strings to Microarray Assay is Predicted to Reduce Noise

For example, the protocol for microarray detection taught in Microarray technology is described in Agilent Low RNA Input Linear Amplification Kit Protocol, Version 4.0, Manual Part No. 5185-5818 (January 2006) is modified according to the present invention by adding nucleic acid strings to the assay. All steps are followed as disclosed in the protocol, except as modified below. In the Agilent oligo protocol for array sample preparation, one adds the population of prepared unlabeled nucleic acid string fragments prior or during step 2 of the labeling protocol. Step 2 uses the template created in step 1 and a T7 RNA polymerase to produce labeled cRNA copies of the original sample mRNA. Typically, 500 ng of total sample RNA is required for microarray analysis. The amount of nucleic acid strings depends on the amplification value. For 1-fold copies, about 500 ng of nucleic acid strings may be used. In general for X-fold amplification approximately X times 500 ng may be used. For large X values, the fragments optionally could be continuously fed into the labeling reaction as needed. The final labeled solution including the plurality of nucleic acid strings would be added to the array during hybridization of the target to the probe on the microarray where the nucleic acid strings improve performance and efficiency of correct probe binding.

It is believed that the signal-to-noise ratio performance, signal accuracy, and speed of the microarray assay will improve significantly by application of the invention.

### Example 2

### Adding Nucleic Acid Strings to BDNA Assay is Predicted to Reduce Noise

For example, the protocol for bDNA assay taught in Collins, et al., A branched DNA signal amplification assay for quantification of nucleic acid targets below 100 molecules/ml, Nucleic Acids Research, 25(15): 2979-2984 (1997) is modified according to the present invention by adding nucleic acid strings to the assay. All steps are followed as disclosed in the protocol, except as modified below. In the protocol for of Collins, one adds the population of prepared unlabeled nucleic acid string fragments prior or during capture of the target onto the capture probe/capture extender complex affixing the target to the microwell. 500 ng of the plurality of nucleic acid strings may be added at this time.

It is believed that the signal-to-noise ratio performance, signal accuracy, and speed of the bDNA assay will improve significantly by application of the invention.

### Example 3

### Adding Nucleic Acid Strings to Nucleic Acid Synthesis

For example, the protocol for PCR taught in Einstein, B. I., “The polymerase chain reaction: a new method of using molecular genetics for medical diagnosis,” New England J. of Med. 322:178-183 (1990) is modified according to the present invention by adding nucleic acid strings to the reaction. All steps are followed as disclosed in the protocol, except as modified below. In the protocol for of Einstein, one adds the population of prepared unlabeled nucleic acid string fragments prior or along with the addition of the polymerase enzyme. 500 ng of the plurality of nucleic acid strings may be added at this time.

It is believed that the efficiency and accuracy of the PCR reaction will be improved.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supercede any such material. The citation of any references herein is not an admission that such references are prior art to the present invention.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

It must be noted that as used herein and in the appended claims, the singular forms “a,” “an,” and “the” include plural referents unless the context clearly dictates otherwise. Thus, for example, reference to “a probe” includes a plurality of such probes and reference to “the sample” includes reference to one or more samples and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

What is claimed is:

1. A method of reducing noise and increasing efficiency in a nucleic acid assay of a biological sample comprising:
(a) providing a plurality of nucleic acid strings;
(b) combining the plurality of nucleic acid strings with an assay to determine the presence, absence, or amount a target nucleic acid; and
(c) allowing the plurality of nucleic acid strings to bind nonspecifically to nucleic acids in the assay, thereby reducing noise and increasing efficiency.

2. The method of claim 1, wherein the nucleic acid assay is a branched DNA assay.

3. The method of claim 1, wherein the nucleic acid assay is an assay conducted on a microarray.

4. The method of claim 1, wherein 90% of the nucleic acid strings in the plurality of nucleic acid strings are from 8 to 70 nucleotides long.

5. The method of claim 1, wherein the plurality of nucleic acid strings is prepared by using restriction enzymes to digest target nucleic acid.

6. The method of claim 1, wherein the plurality of nucleic acid strings is prepared by using restriction enzymes to digest complement of the target nucleic acid.

7. The method of claim 1, wherein the plurality of nucleic acid strings is prepared by using restriction enzymes to digest nucleic acids in an aliquot of the biological sample.

8. The method of claim 1, wherein the plurality of nucleic acid strings is prepared randomly on an oligonucleotide synthesizer.

9. The method of claim 1, wherein the plurality of nucleic acid strings is prepared on an oligonucleotide synthesizer based on a known sequence.

10. The method of claim 9, wherein the known sequence is fragments of complement of the target nucleic acid.

11. A method of increasing the accuracy and efficiency of a nucleic acid synthesis reaction comprising:
(a) providing a plurality of nucleic acid strings;
(b) combining the plurality of nucleic acid strings with a nucleic acid synthesis reaction comprising a template nucleic acid, synthetic and/or natural nucleic acids, and an enzyme for nucleic acid synthesis; and
(c) allowing the plurality of nucleic acid strings to bind nonspecifically to the products of the nucleic acid synthesis reaction, thereby increasing the accuracy and efficiency of a nucleic acid synthesis reaction.

12. The method of claim 11, wherein the nucleic acid synthesis reaction comprises transcription of DNA to RNA using a polymerase.

13. The method of claim 12, wherein the polymerase is a T7 RNA polymerase.

14. The method of claim 11, wherein the nucleic acid synthesis reaction comprises replication of RNA, DNA, or synthetic nucleic acids.

15. The method of claim 11, wherein the nucleic acid synthesis reaction comprises amplification of RNA, DNA, or synthetic nucleic acids.

16. The method of claim 15, wherein the amplification reaction is PCR.

17. The method of claim 11, wherein the nucleic acid synthesis reaction is a reverse transcription reaction.

18. The method of claim 11, wherein 90% of the nucleic acid strings in the plurality of nucleic acid strings are from 8 to 30 nucleotides long.

19. The method of claim 11, wherein the plurality of nucleic acid strings is prepared by using restriction enzymes to digest the template nucleic acid.

20. The method of claim 11, wherein the plurality of nucleic acid strings is prepared by using restriction enzymes to digest complement of the template nucleic acid.

21. The method of claim 11, wherein the plurality of nucleic acid strings is prepared by using restriction enzymes to digest a biological sample.

22. The method of claim 11, wherein the plurality of nucleic acid strings is prepared randomly on an oligonucleotide synthesizer.

23. The method of claim 11, wherein the plurality of nucleic acid strings is prepared on an oligonucleotide synthesizer based on a known sequence.

24. The method of claim 23, wherein the known sequence is fragments of the template nucleic acid.

25. The method of claim 23, wherein the known sequence is fragments of complement of the template nucleic acid.
